(19) United States
(12) Patent Application Publication
(10) Pub. No.: US 2017/0138960 A1
(43) Pub. Date: May 18, 2017

(54) DETECTION, MONITORING AND TREATMENT OF ACUTE MYOCARDIAL INFARCTION

(71) Applicant: DIADEXUS, INC.

(72) Inventor: Shaoqiu ZHUO, Moraga, CA (US)

(21) Appl. No.: 15/115,750

(22) PCT Filed: Feb. 2, 2015
(86) PCT No.: PCT/US15/14140

§ 371 Date: Aug. 1, 2016

Related U.S. Application Data
(60) Provisional application No. 61/934,666, filed on Jan. 31, 2014.
(60) Provisional application No. 61/938,088, filed on Feb. 10, 2014.

(51) Int. Cl. G01N 33/68; G01N 33/543; A61K 33/40; G01N 33/58

(52) U.S. Cl. CPC G01N 33/6893; G01N 33/581; G01N 33/54306; G01N 33/5436; A61K 33/40; G01N 2333/775; G01N 2333/92; G01N 2800/324

(57) ABSTRACT

Detection, monitoring and treatment of acute myocardial infarction (AMI) using a hybrid immunoassay to both Lp-PLA2 and ApoA1. Described herein are assays and method of performing them useful for the detection of AMI. Also described are methods of monitoring a patient at risk for, or suffering from, AMI. Also described herein are methods and assays for treating patient having or at risk of suffering from AMI.

### CROSS REFERENCE TO RELATED APPLICATIONS

This patent application claims priority to U.S. provisional patent application no. 61/934,666, filed Jan. 31, 2014 (“DETECTION, MONITORING AND TREATMENT OF ACUTE MYOCARDIAL INFARCTION”) and U.S. provisional patent application No. 61/938,088, filed Feb. 10, 2014 (“DETECTION, MONITORING AND TREATMENT OF ACUTE MYOCARDIAL INFARCTION”).

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are systems, including assays, and methods for the detection of cardiovascular conditions and diseases, and more specifically, systems and methods for the detection and monitoring of acute myocardial infarction. For example, the systems and methods described herein may provide a method for ruling out acute myocardial infarction (AMI) in a patient presenting with chest pain suspected to be cardiac in nature. These methods may include treatment of the patient based on the determination.

### BACKGROUND

Each year over a million people in the U.S. have a heart attack, also known as a myocardial infarction. About half of them die as a result. Many people have permanent heart damage or die because they don't get help immediately. The symptoms of a heart attack include chest discomfort such as pressure, squeezing, or pain; shortness of breath; discomfort in the upper body including the arms, shoulders, neck, and back; nausea; vomiting; dizziness; lightheadedness; and sweating. Most heart attacks happen when a clot in the coronary artery blocks the supply of blood and oxygen to the heart. Often this leads to an irregular heartbeat, called an arrhythmia, that causes a severe decrease in the pumping function of the heart. A blockage that is not treated within a few hours causes the affected heart muscle to die.

Acute myocardial infarction (AMI) is the death or necrosis of myocardial cells, caused by the interruption of the blood supply to the heart. Much of the damage associated with AMI is due to contraction band necrosis. However, it is now recognized that both apoptosis and necrosis contribute to the myocardial damage seen in patients with AMI with apoptosis contributing to up to 50% of the overall injury. In patients who die after AMI, apoptosis is present in both regions adjacent to and remote from the infarction. Further, while timely reperfusion of the ischemic myocardium can limit infarct size, reperfusion itself may cause damage to the previously ischemic myocardium, including augmentation of the apoptosis which occurs during occlusion. The number of apoptotic cells in the perinecrotic myocardium progressively increases during reperfusion, contributing substantially to the overall extent of the infarction.

Cardiac biomarkers have revolutionized the care of cardiovascular patients in numerous arenas, including prediction and detection of pre-clinical disease, improved detection of cardiac injury including non-ST-segment-elevation myocardial infarction (NSTEMI), prognostication in both acute and chronic disease presentations, and monitoring the response to treatment. Most biomarkers, however, are markers of necrosis. There remains a need for new biomarkers, specifically a biomarker that can detect and quantify early and accurately, suitable detecting and/or diagnosing acute myocardial infarction.

Further, the search for an effective strategy that would help clinicians to exclude the diagnosis of acute myocardial infarction (AMI) without the need for serial troponin testing over a number of hours has been ongoing for many years. High sensitivity troponin (hs-Tn) assays, which have greater analytical sensitivity and precision than standard assays, have been shown to improve sensitivity for AMI when measured at the time of initial presentation. However, while the negative predictive value is improved at the time of presentation, even hs-Tn cannot exclude AMI without serial sampling. Studies have also shown that hs-Tn assays can detect cardiac troponin in patients with stable heart disease who have not suffered an acute event. Troponin is a ‘late marker’ of myocardial necrosis (blood levels may take several hours to increase significantly), and has been significant interest in using so-called ‘early markers’ of myocardial necrosis to exclude AMI during the period of ‘troponin blindness’.

### SUMMARY OF THE DISCLOSURE

In general, described herein are LpPLA2/ApoA1 assays (referred to herein as hybrid assays and hybrid LpPLA2/ApoA1 assays, method of performing the assays, and methods of treating a patient using the information determined from the assays that may be particularly sensitive to treatment (including ruling out or ruling in) myocardial infarction (AMI).

For example, described herein are methods of treating, detecting or monitoring acute myocardial infarction (AMI) comprising: exposing a blood sample from a patient to a solid phase support onto which an Lp-PLA2-binding molecule is coupled; incubating the blood sample with an ApoA1-binding molecule; and detecting the level of ApoA1 from the solid phase.

A method of treating, detecting or monitoring acute myocardial infarction (AMI) may include: exposing a blood sample from a patient to a solid phase support onto which an Lp-PLA2-binding molecule is coupled; incubating the solid phase support with an ApoA1-binding molecule; washing the solid phase support; detecting the level of ApoA1 from the solid phase; and treating the patient for AMI if the amount of ApoA1 detected is below a threshold.

In any of the methods and systems described herein, any marker for HDL may be used in place of (or in addition to) ApoA1.

Also, any of the method of treatment described herein may include treating the patient for AMI if the level of ApoA1 (or some other marker of HDL) is below a threshold. For example, a method of treating a patient for AMI if the level of ApoA1 detected is below 0.7 ng/ml (e.g., below 0.71, 0.72, 0.73, 0.74, 0.75, 0.76, 0.77, 0.78, 0.79, 0.8, 0.81, 0.82, 0.83, 0.84, 0.85 ng/ml, etc.).

Any of these methods may include washing the solid phase support during the methods described. Washes may be performed with solutions (e.g., pH buffered, salt-balanced, solutions as described herein). In general, the step of binding to the solid phase support may be performed prior to exposure to the ApoA1 binding moiety, and the solid phase support may be washed first, following incubation with the sample. A sample may be any tissue sample, including in particular a blood (e.g., plasma, whole blood, etc.) sample.

Exposing the sample from the patient to the solid phase support may generally comprise adding the sample to a solid phase support to which an antibody or an antibody fragment specific for Lp-PLA2 is coupled. The sample may be incubated with the solid phase for any appropriate amount of time (e.g., seconds, minutes, etc.).

Any solid phase support may be used, including in particular, surfaces of coverslips, wells (e.g., multi-well plates), beads (e.g., particles), strips (including paper/polymeric supports), etc.

Incubating the blood sample with the ApoA1-binding molecule may comprise incubating with an antibody or an antibody fragment that binds ApoA1.

Any detection method may be used for detecting the level of ApoA1 from the solid phase support, including optical (e.g., immunoflorescent, etc.), radioactivity, enzymatic (e.g., HRP, etc.). For example, detection may comprises reacting a horseradish peroxidase (HRP) bound to an antibody against ApoA1 (or to a secondary antibody binding to the anti-ApoA1) to detect a signal and quantifying the detected signal.

Also described herein are systems for treating and/or detecting and/or monitoring AMI. For example, a system for treating, detecting, and/or monitoring acute myocardial infarction (AMI) may include: a first component comprising an Lp-PLA2 binding molecule coupled to a solid-phase support; a reagent comprising an ApoA1 binding molecule to which a detection moiety is coupled.

For example, a system for treating, detecting or monitoring acute myocardial infarction (AMI) may include: a first component comprising an LpPLA2 binding molecule coupled to a solid-phase support; a first wash buffer; a reagent comprising an ApoA1 binding molecule to which a detection moiety is coupled; and a detection buffer.

The first component may comprise a solid-phase support to which an antibody or antibody fragment that binds to Lp-PLA2 has been coupled. The reagent may comprise a solution including an ApoA1 antibody or antibody fragment coupled to a detection moiety. The system may also include a set of standards configured to calibrate the level of ApoA1 detected by the detection moiety.

### BRIEF DESCRIPTION OF THE DRAWINGS

FIG. 1 is a schematic overview of one variation of a hybrid LpPLA2/ApoA1 assay for use in monitoring, detecting and/or treating a cardiac disorder such as (but not limited to) AMI. FIG. 1 shows an ELISA Format Design for an Lp-PLA₂/ApoAI Hybrid Assay.

FIG. 2 (which includes FIGS. 2A-2F) shows results of a hybrid LpPLA2/ApoA1 assay, examining both control and AMI patients. FIG. 2A shows a one-way analysis of APoA1 (ng/ml) by AMI for the Lp-PLA₂/ApoAI Hybrid Assay-ANOVA Analysis. FIG. 2B shows the summary of fit for the one way ANOVA, FIG. 2C shows the t-test results, FIG. 2D shows the analysis of variance, FIG. 2E shows the means for the one way ANOVA, and FIG. 2F shows the means comparisons.

FIG. 3 (which includes FIGS. 3A-3D) shows a logistic fit for the results of the hybrid LpPLA2/ApoA1 assay of FIG. 2. FIG. 3A shows a logistic fit of AMI by ApoA1 (ng/ml) of the Lp-PLA₂/ApoAI Hybrid Assay-Logistic Fit. FIG. 3B shows a whole-model test, FIG. 3C shows Parameter estimates, and FIG. 3D shows the receiver operating characteristic (ROC) curve.

FIG. 4 (which includes FIGS. 4A-4F) shows results of an LpPLA2 assay, examining both control and AMI patients. FIG. 4A shows a one-way analysis of PLC mass by AMI for the Lp-PLA₂ Mass Assay (PLAC)-ANOVA Analysis. FIG. 4B shows the summary of fit for the one way ANOVA, FIG. 4C shows the t-test results, FIG. 4D shows the analysis of variance, FIG. 4E shows the means for the one way ANOVA, and FIG. 4F shows the means comparisons.

FIG. 5 (which includes FIGS. 5A-5D) shows a logistic fit for the results of the LpPLA2 assay of FIG. 4. FIG. 5A shows a logistic fit of AMI by PLAC mass of the Lp-PLA2 Mass (PLAC) Assay-Logistic Fit. FIG. 5B shows a whole-model test, FIG. 5C shows Parameter estimates, and FIG. 5D shows the receiver operating characteristic (ROC) curve.

FIG. 6 (which includes FIGS. 6A-6F) shows results of an ApoA1 assay, examining both control and AMI patients. FIG. 6A shows a one-way analysis ApoA1 (mg/dL) by AMI for the ApoA1 Assay-ANOVA Analyses. FIG. 6B shows the summary of fit for the one way ANOVA, FIG. 6C shows the t-test results, FIG. 6D shows the analysis of variance, FIG. 6E shows the means for the one way ANOVA, and FIG. 6F shows the means comparisons.

FIG. 7 (which includes FIGS. 7A-7D) shows a logistic fit for the results of the ApoA1 assay of FIG. 6. FIG. 7A shows a logistic fit of AMI by ApoA1 (ng/ml) of the ApoAI Assay-Logistic Fit. FIG. 7B shows a whole-model test, FIG. 7C shows Parameter estimates, and FIG. 7D shows the receiver operating characteristic (ROC) curve.

FIG. 8 (including FIGS. 8A-8C) is a comparison of the logistic fits of the hybrid Lp-PLA2/ApoA1, LpPLA2 and ApoA1 assays. FIG. 8A is a logistic fit of AMI by ApoA1 (ng/ml). FIG. 8B is a logistic fit of AMI by PLAC mass. FIG. 8C is a logistic fit of AMI by ApoA1.

FIG. 9A shows comparisons of the ROC curves for the hybrid Lp-PLA2/ApoA1 (FIG. 9A1), LpPLA2 (FIG. 9A2) and ApoA1 assays (FIG. 9A3).

FIGS. 9B-9D provide further detail on the data illustrated in FIG. 9A. FIG. 9B shows the ROC curve analysis of the Lp-PLA2 Mass/ApoA1 hybrid assay; FIG. 9C shows the ROC curve analysis of the Lp-PLA2 (PLAC) mass assay; and FIG. 9D shows the ROC curve analysis of the ApoA1 assay.

FIG. 10 (including FIGS. 10A-10E) illustrates the results of an ApoA1 assay for CVD (cardiovascular disease). FIG. 10A graphically depicts a one-way analysis of ApoA1 by CVD. FIG. 10B shows the one-way analysis, including the summary of fit, t-test, analysis of variance, and means for one-way ANOVA. FIG. 10C shows the means comparison. FIG. 10D shows a logistic fit of CVD by ApoA1. FIG. 10E shows the ROC curve.

FIG. 11 (including FIGS. 11A and 11B) is a comparison of the logistic fits of the hybrid LpPL2/ApoA1 assay for AMI and the ApoA1 assay for CVD (of FIG. 10). FIG. 11A shows the logistic fit of AMP by ApoA1 using plasma for the Lp-PLA₂ Mass/ApoAI Hybrid Assay. FIG. 11B shows a logistic fit of CVD by ApoA1 for an Lp-PLA₂ Mass/ApoAI Hybrid Assay using serum.

FIG. 12A (including FIGS. 12A1 and 12A2) shows an ANOVA Analysis of another set of control patients compared to the AMI patients and previous control data. FIG. 12A1 shows the one-way analysis of ApoA1 conc. by group. FIG. 12A2 shows the details of the one-way analysis, including the summary of fit, analysis of variance, means for the one-way ANOVA, and means comparison.

FIG. 12B (including FIGS. 12B1-12B3) shows a logistic fit for the results of the hybrid LpPLA2/ApoA1 assay of FIG. 12A. FIG. 12B1 shows the logistic fit of AMI by ApoA1 conc. FIG. 12B2 shows the whole model test data and parameter estimates. FIG. 12B3 shows the ROC curve.

FIG. 13 (including FIGS. 13A and 13B) shows the ROC curve for the hybrid (LpPLA2/ApoA1) assay (FIG. 13A), and indicates the Youden cutoff criterion (e.g., ≦0.803 ng/ml) calculated from the ROC (FIG. 13B).

### DETAILED DESCRIPTION

A hybrid Lp-PLA₂/ApoAI Hybrid Assay was designed and constructed to compare blood samples from both acute myocardial infarction (AMI) and non-symptom control (Caucasian patients). In general the assay systems described herein are configured to sequentially pull down first an LpPLA2 binding component of a biological sample (e.g., blood, plasma, serum, whole blood, etc.) and then probe for ApoA1 from the bound LpPLA2 component.

The assays described herein my typically include a solid phase component that includes specific binding to LpPLA2 from a body fluid (e.g., blood, etc.). The assay may also include one or more wash buffers. Further, the assay may then include a labeled probe for ApoA1.

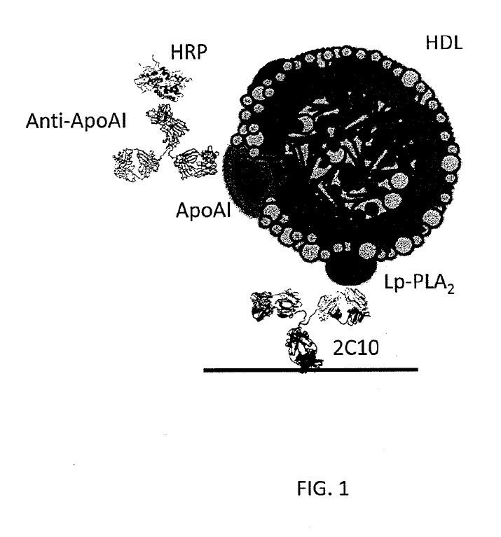

For example, FIG. 1 schematically illustrates one possible principle for operation of the assays described herein. In this example, an LpPLA2-binding molecule is coupled to a solid phase support (e.g., a well or plate). The Lp-PLA2 binding molecule is an antibody (e.g., 2C10) to Lp-PLA2 is linked/absorbed to a solid phase surface (e.g., a well or wells of a microtiter plate, etc.). Any appropriate antibody capable of binding to Lp-PLA2 may work, including, for example, those described in U.S. patent application no. US-2014-0283157-A1, herein incorporated by reference in its entirety. A biological solution (e.g., blood) is then added to the solid phase and LpPLA2 from the sample is allowed to bind. The solid phase may then be washed, then probed with a detectable molecule that binds to ApoA1 (e.g., “ApoA1-binding molecule”); in this example, the Apo-A1 binding molecule is an antibody specific to Apolipoprotein A-I (ApoA1). ApoA1 is the major protein component of high density lipoprotein (HDL) in plasma. Thus, in some variations, other components of HDL (or other lipoproteins) may be used. The ApoA1 binding molecule may then be detected by either direct detection (e.g., where the APOA1 binding molecule is labeled, e.g. fluorescently labeled, HRP labeling, etc.) or detected by a secondary marker that specifically targets the ApoA1 binding molecule.

The amount of HDL is then assayed. For example, the ApoA1 binding molecule may be labeled, marked, or coupled to a marker/label, as mentioned. In FIG. 1, the ApoA1 antibody to the HDL component includes an indicator, in this example, horseradish peroxidase (HRP), that may be reacted to detect a signal. In some variations the marker may be directly visualized (e.g., fluorescent, etc.).

In one specific example, the Lp-PLA2/ApoAI Hybrid Assay protocol included: an LpPLA2 assay (e.g., commercial PLAC mass assay kit was manufactured by diaDexus, Inc.) modified for use with an ApoAI assay kit manufactured by AlerCheck, Inc. (15 Oak St., Ste 302, Springvale, Me. 04083).

In use, 20 μl each of human plasma samples were loaded onto a 2C10 coated 96-well (from PLAC mass assay) and incubated at room temperature for 10 minutes. As a calibration/control, 4 strips of PLAC mass assay kit were replaced with that from the ApoA1 ELISA kit and 20 μl of the ApoA1 standard (with series dilution from 36 ng/ml to 0 ng/ml in 13 points) in duplication were added. 100 μl of HRP conjugated affinity purified goat anti-ApoAI (from AlerCheck ApoAI ELISA kit) were added into each well and incubated at room temperature for 2 hr. After washed the plate with TBST (tris buffered saline, pH 7.2, with 0.005% Tween-20 from PLAC mass assay kit), the plate was incubated with 100 μl of TMB (from PLAC mass assay kit) for 30 minutes at room temperature and stopped with 100 μl of 1 M HCl (from PLAC mass assay kit). Signal was read at 450 nm.

The plasma samples used included both acute myocardial infarction (AMI) patient's (positive) and non-symptom controls; all patients and controls were Caucasian. For all of the AMI patients, the AMI events are first time episode. Blood were collected between 7 and 12 hr. after symptom onset. All plasmas are EDTA plasmas. AMI cases were confirmed by electrocardiogram and the elevation of troponin-1 level (AQT90FLEX—immuno-chemical analyzer/Radiometry). AMI plasmas were collected during May to June, 2010 and control plasmas were collected during January to May, 2013.

TABLE 1
Information of AMI Plasma and Control
Information on blood samples for AMI and control samples

| Sample ID | Female | Male | Age | Height | Weight | BMI |
|---|---|---|---|---|---|---|
| Control | 13 (62%) | 8 (38%) | 73 | 171 | 79 | 27 |
| AMI | 15 (60%) | 10 (40%) | 73 | 168 | 75 | 27 |

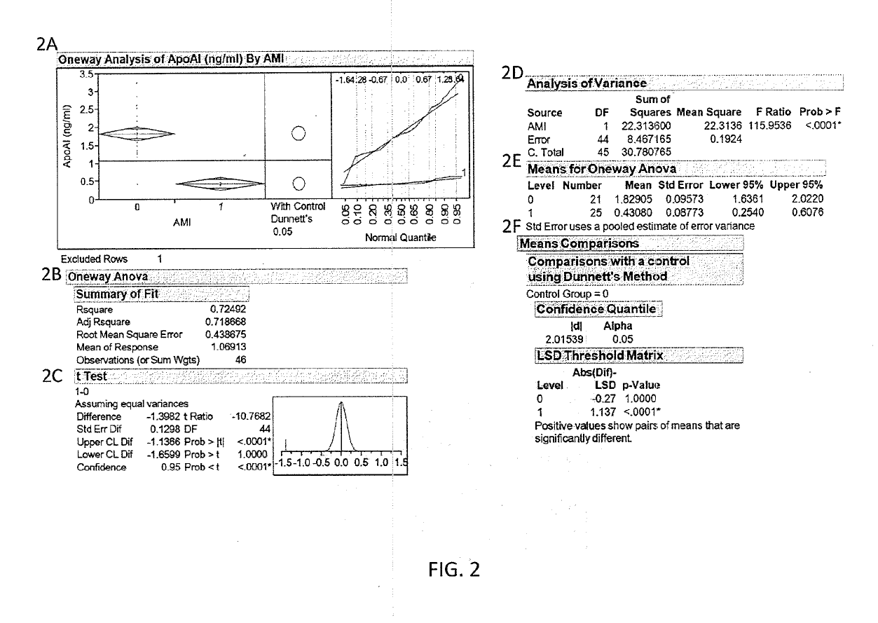

Results from the analysis, comparing signals between control and AMI patients for LpPLA2 alone, ApoA1 alone, and the hybrid LpPLA2/ApoA1 assay are shown in data (including graphs) in FIGS. 2-11.

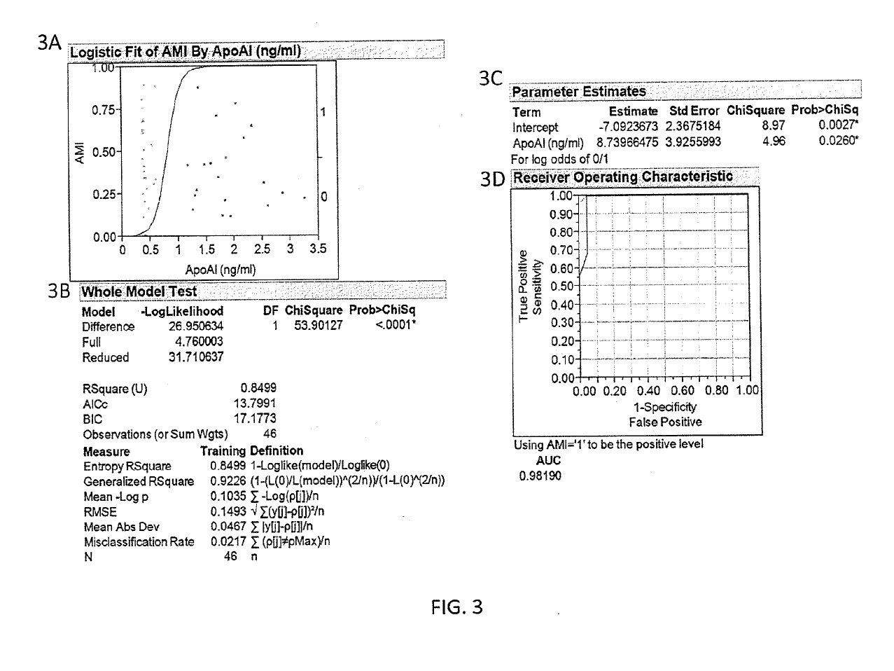

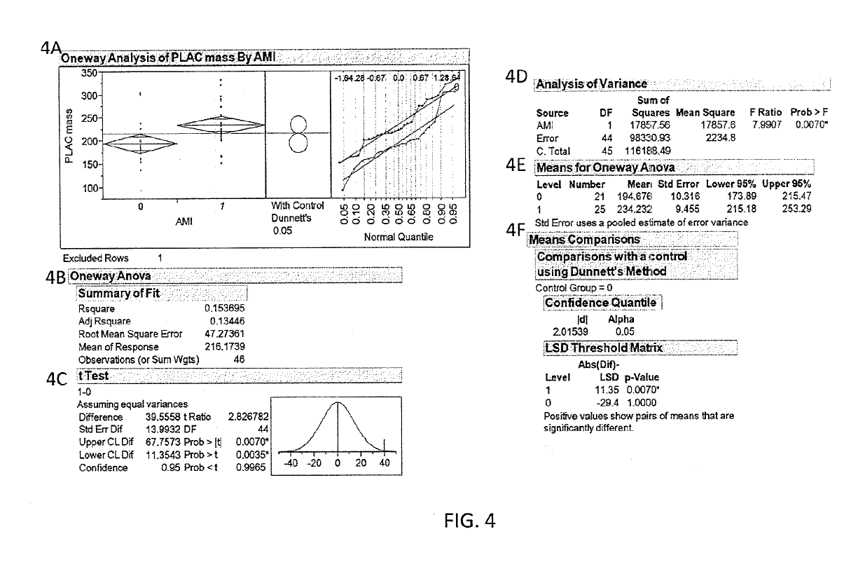

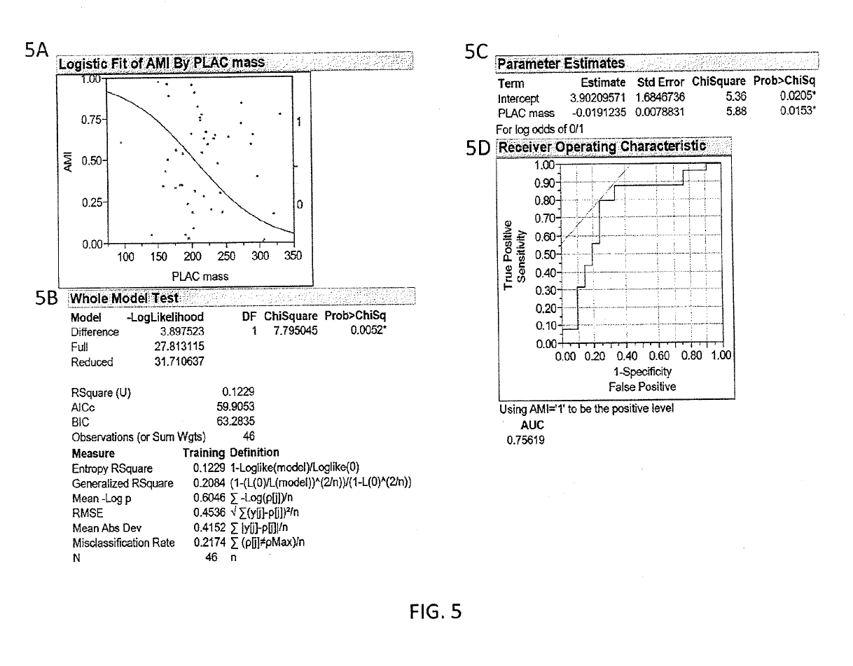

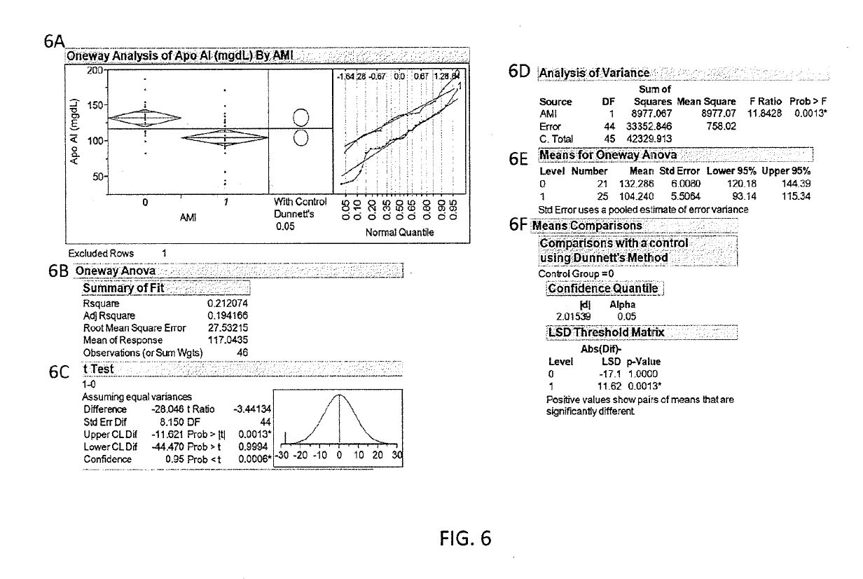

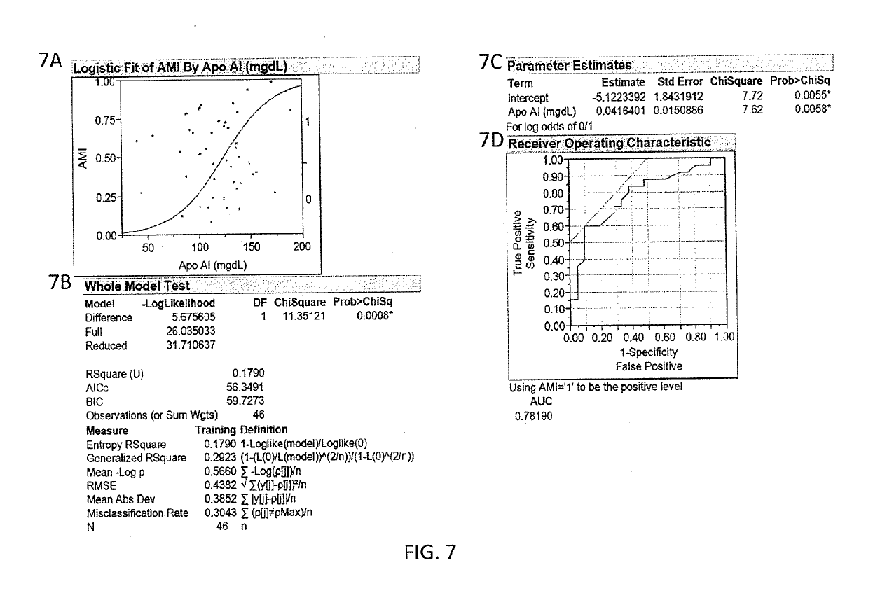

For example, FIGS. 2-3 show preliminary characterization of the results of the hybrid LpPLA2/ApoA1 assay. A clear distinction in the control versus AMI groups may be seen. FIGS. 4-5 illustrate the results of just the LpPLA2 assay of the control and AMI samples groups. FIGS. 6-7 illustrate the results characterizing just the ApoA1 assay in the same AMI and control individuals.

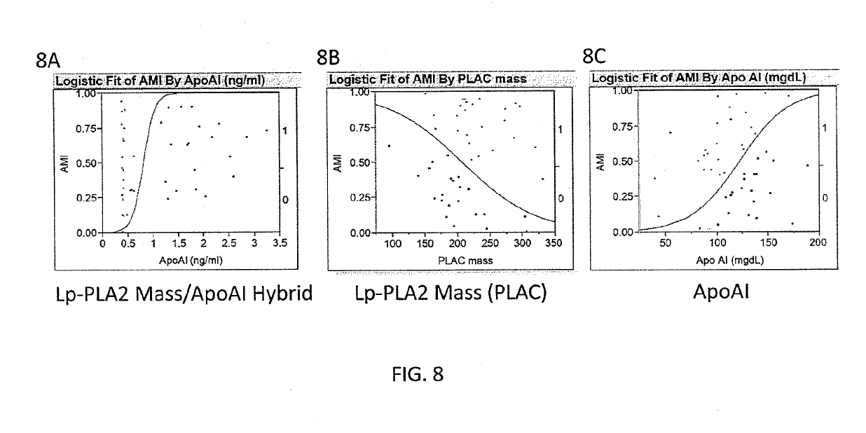

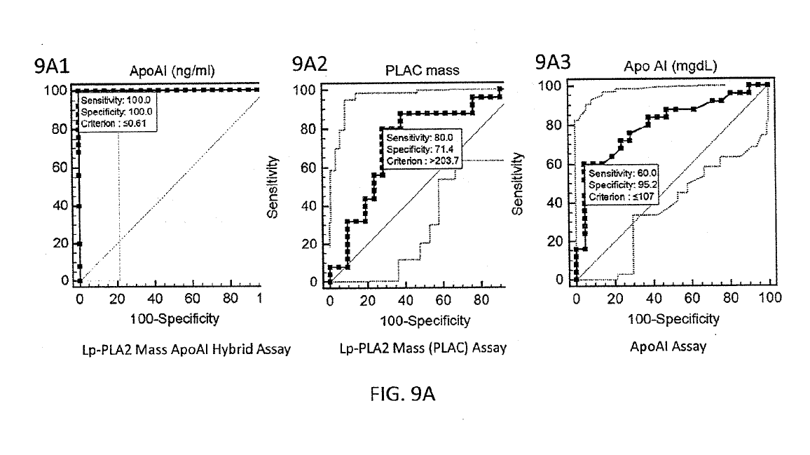

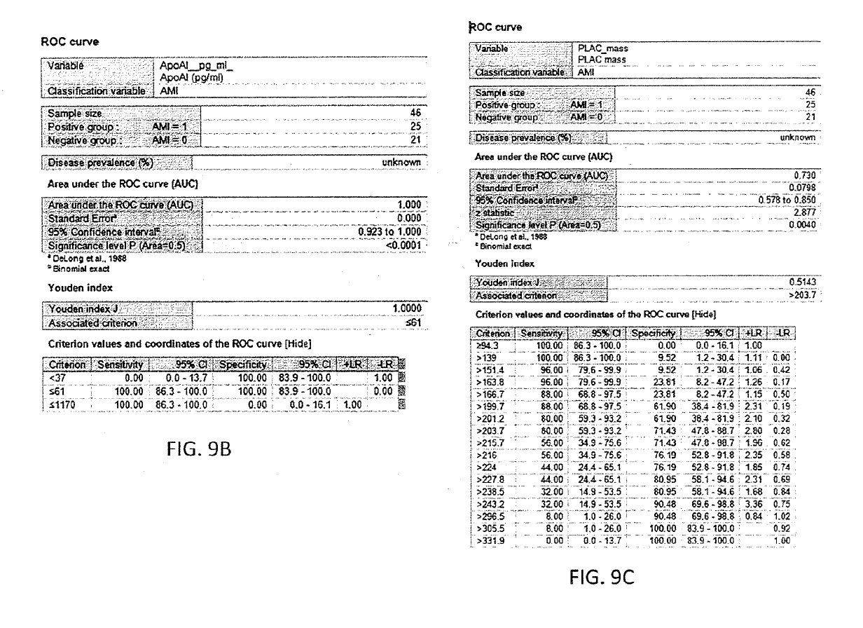

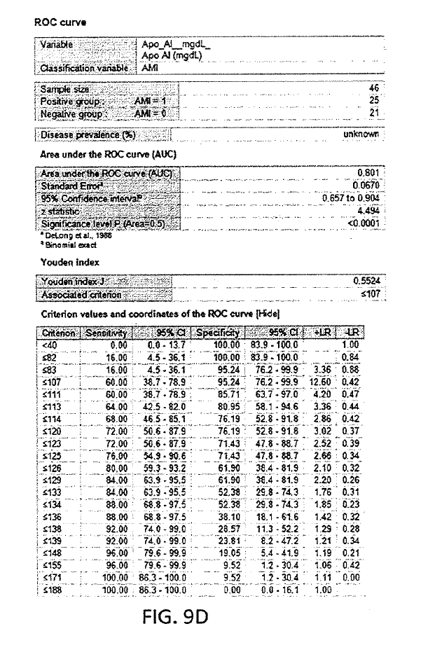

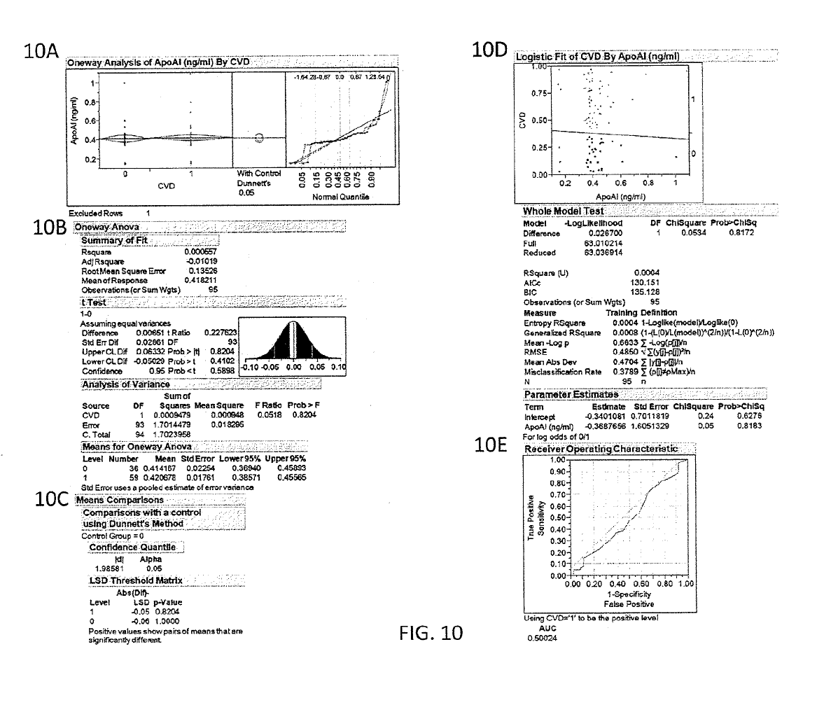

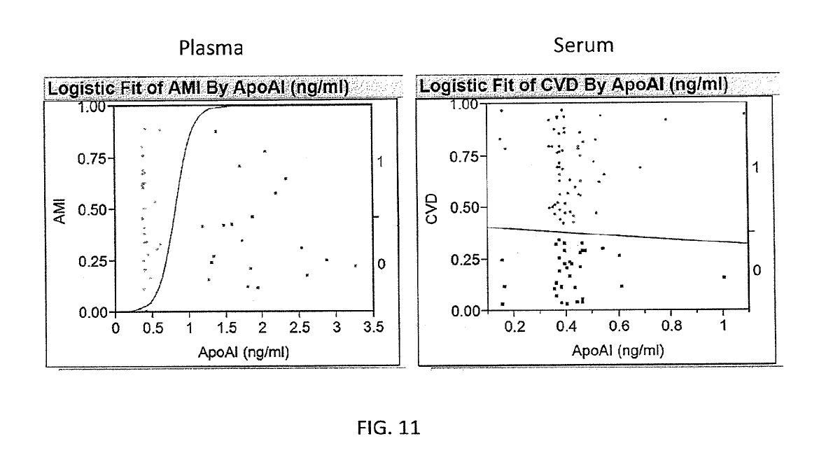

Individually, ApoA1 and LpPLA2 may provide some distinctions between the two groups, however, the combined/hybrid assay provides a remarkably robust separation of AMI and control (non-AMI) groups. For example, FIG. 8 compares the logistic fit between the hybrid (far left), LpPLA2 alone (middle) and ApoA1 (right). The strong separation of AMI and non-AMI groups is even more pronounced when looking at the receiver operator characteristic (ROC) curves and cutoff comparison in FIGS. 9A-9D. As shown in FIG. 9A1, the assay results in an almost idea ROC result (showing both high sensitivity and high specificity) compared to the ROC for Lp-PLA2 mass (PLAC) assay alone or APoA1 assay alone as shown in FIGS. 9A2 and 9A3, respectively. The details of these results are provided in FIGS. 9B-9D. FIG. 10 shows (for comparison) an analysis of ApoA1 by cardiovascular disease. FIG. 11 shows a comparison of plasma and serum samples examined (on left) as described above for the hybrid Lp-PLA2/ApoA1 assay for AMI, compared with a serum assay for cardiovascular disease (CVD) using ApoA1.

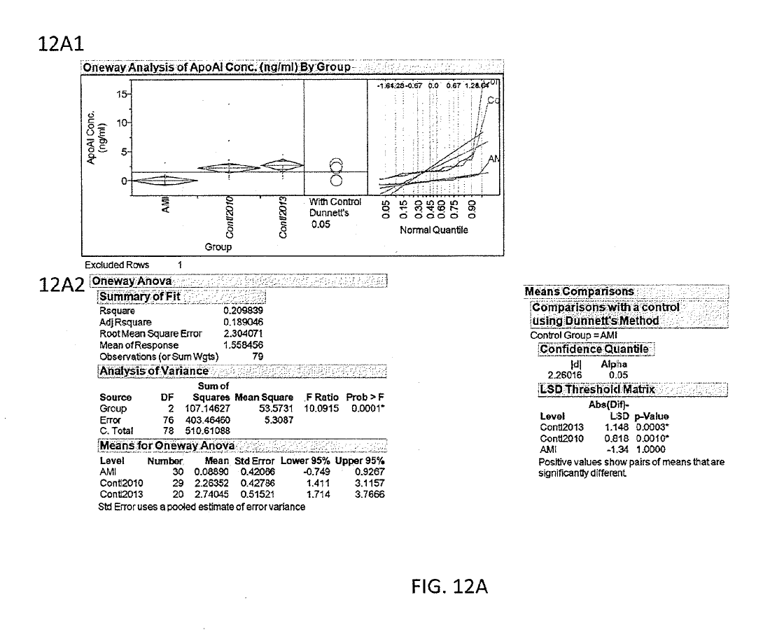

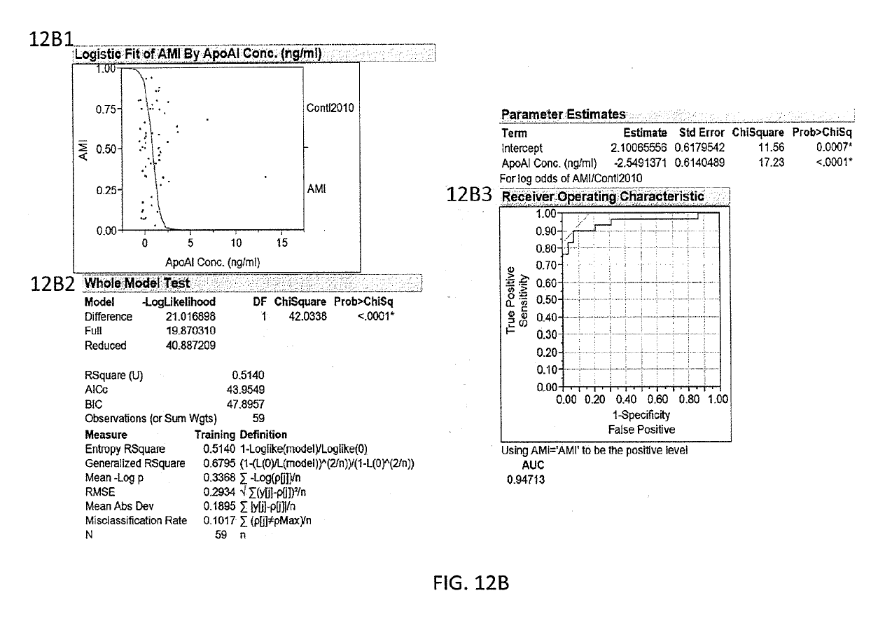

In comparison to the ApoA1 assay and/or the Lp-PLA2 assays alone, the hybrid Lp-PLA2 and ApoA1 assay shows an extremely high degree of specificity and sensitivity. The reliability of the results was confirmed by the ANOVA analysis and logistic fits. In FIGS. 2 and 12, one way ANOVA analysis of the LpPLA2/ApoA1 hybrid assay is shown. The ANOVA results show a good separation between the control (or first control and second control) groups and the AMI group, both within groups and between groups, when looking at the within group error and the between group error. In these cases the error with the groups was smaller than the error between the groups, suggesting a reliable level of separation between the control and AMI groups. Similarly, the logistic fit between the groups suggests that the hybrid (LpPLA2/ApoA1) assay reflects a high degree of separation between the AMI and control groups; the nearly vertical line separating the AMI group from the control (e.g., FIG. 3 Logistic fit of AMI by ApoA1, for the ApoA1 pulled down by the LpPLA2 in the hybrid assay) indicates a high degree of separation. Compare this to the less robust correlation for either LpPLA2 or ApoA1 alone, as shown in FIGS. 4-5 and 6-7, as well as FIG. 8.

In the data shown in FIGS. 2-8, the control samples were more recent than the AMI samples. To confirm that the results were not an artifact due to the age of the control versus the AMI samples, the assay was re-run using controls taken at about the same time (from age/gender matched control patients). The results are shown in FIGS. 12A-12B. In this example, 20 ul of each CVD or control plasma (ProteoGenex) were incubated with 2C10 plate for 10-15 min at room temperature, as described above. Next, 100 ul of anti-ApoAI-HRP conjugate (AlerCHEK ELISA kit, Cat# A70101, Lot# K103414) was added, mixed and the plates were incubated at room temperature for 3 hr. Plates were then washed with PLAC kit wash buffer and detected with reagents of the PLAC test. In addition, plates were incubated with 100 ul/well TMB for 60 min (Plate 4) or overnight (Plate 3) and stopped with 100 ul/well of 1 M HCl.

The results of this second set of experiments confirm the earlier data (e.g., shown in FIGS. 2-8). Specifically, the control groups (“control 2010” and “contro12013”) were nearly identical to each other, and significantly different from the AMI group.

Thus, the results of these experiments indicate that an assay that includes a solid phase linked LpPLA2 binding molecule and a probe for ApoA1 to determine the level of ApoA1 associated with LpPLA2 pulled out of a fluid (e.g., blood) sample from a patient may reliably indicate acute myocardial infraction. Further, this assay may be surprisingly more robust (both specific and sensitive) than either LpPLA2 or ApoA1 alone.

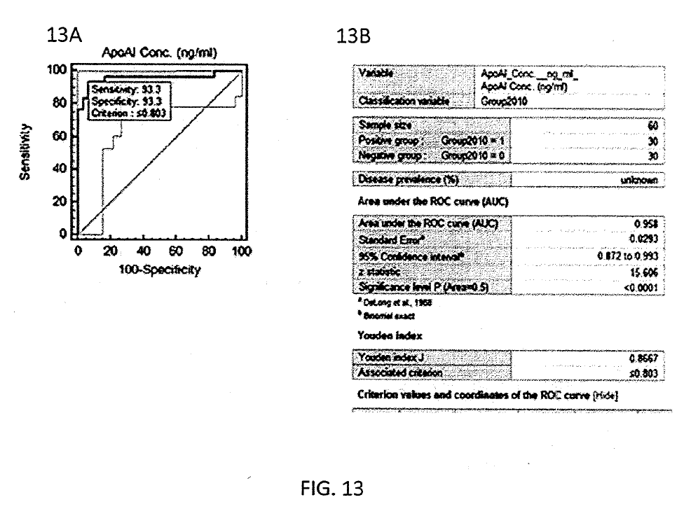

In use, the hybrid (Lp-PLA2/ApoA1) assay may be used to determine if a patient has experienced an AMI. The assay may be performed quickly (e.g., within a few hours) and a single result may be indicative. Thus, for example, if the level of ApoA1 from a sample LpPLA2 bound biological (blood) material) is below a threshold value, the patient has likely experienced (or is experiencing) and acute myocardial infarct (AMI) event. Any appropriate threshold may be used. In particular, the threshold may be determined from the ROC curve, as illustrated in FIG. 13. In this example, a cutoff value (e.g., the Youden cutoff criterion) has been determined to be approximately ≦0.803 ng/ml. Thus, for example, if the level of ApoA1 from a sample of LpPLA2 bound material from a patient's fluid sample is less than or equal to the cutoff (e.g., approx. 0.8) then the patient is positive for AMI (e.g., likely experienced an AMI). For example, the cutoff may a level of ApoA1 detected from a sample pulled-down with Lp-PLA2 (using an Lp-PLA2 solid phase support) that is less than about 0.7 ng/ml, e.g., below about 0.72 ng/ml, below about 0.73 ng/ml, below about 0.74 ng/ml, below about 0.75 ng/ml, below about 0.76 ng/ml, below about 0.77 ng/ml, below about 0.78 ng/ml, below about 0.79 ng/ml, below about 0.8 ng/ml, below about 0.81 ng/ml, below about 0.82 ng/ml, below about 0.83 ng/ml, etc.).

In addition, the construction of the assay has also been found to be important in determining AMI risk and treatment. For example, when ApoA1 or other non-Lp-PLA2 components of the HDL were used to first tether the HDL, which was then probed for Lp-PLA2 (e.g., using any of the same components described herein, including 2C10), there was no or only weak correlation between detected signal and AMI (data not shown). This was both surprising and informative, given the strength of the signal when the hybrid assay is performed as described above. Thus, is important that the steps of performing the assays are executed in the proper order, so that the Lp-PLA2 is used to initially pull-down the HDL particle/fraction, which is thereafter probed for ApoA1. Note that it may be possible to label the ‘loose’ (untethered) HDL particles (e.g., bound to labeled ApoA1) first, before tethering them with a solid-phase to Lp-PLA2, however, based on the current data it appears important that the Lp-PLA2 be used as the solid-phase (tethering) link.

### Treatment

Morbidity and mortality from myocardial infarction are significantly reduced if recognized early, so that prompt attention may be provided, and to shorten the time to definitive treatment. It may also be beneficial to recognize that an AMI has occurred within the recent past (e.g., within 1-24 hours). As a general rule, initial therapy for acute myocardial infarction is directed toward restoration of perfusion as soon as possible to salvage as much of the jeopardized myocardium as possible. This may be accomplished through medical or mechanical means, such as PCI or CABG. Treatment may also include restoration of the balance between the oxygen supply and demand to prevent further ischemia, pain relief, prevention and treatment of any complications that may arise, and coronary collateral circulation.

Thus, in general, described herein are treatment methods that include performing a hybrid LpPLA2/ApoA1 assay (or any other hybrid LpPLA2/HDL assay) to determine if the amount of ApoA1 selectively pulled down by LpPLA2 solid phase is low compared to a threshold (e.g., is below a threshold, such as below 0.7 ng/ml, e.g., below 0.72 ng/ml, below 0.73 ng/ml, below 0.74 ng/ml, below 0.75 ng/ml, below 0.76 ng/ml, below 0.77 ng/ml, below about 0.78 ng/ml, below 0.79 ng/ml, below about 0.8 ng/ml, below about 0.81 ng/ml, below 0.82 ng/ml, below 0.83 ng/ml, etc.). If below this threshold, the patent may be experiencing (or may have recently experienced) AMI and should be treated for AMI, for example, by delivering reperfusion therapy.

In some variations, the patient may be hospitalized and may immediately receive one or more of: oxygen (e.g., by nasal prongs); sublingual nitroglycerin (e.g., unless systolic arterial pressure is less than 90 mm Hg or heart rate is less than 50 or greater than 100 beats per minute); analgesia (e.g., with morphine sulfate or meperidine); aspirin (e.g., 160 to 325 mg orally); and/or immediate reperfusion therapy, either by fibrinolysis or primary percutaneous transluminal coronary angioplasty (PTCA).

Treatment of AMI may therefore generally be directed to preventing ischemia, relieving pain, and preventing complications of AMI. Treatments may include pharmacological treatment (e.g., using drugs such as aspirin), procedural therapies (e.g., reperfusion therapies), and monitoring. For example, a patient may be monitored by electrodes (EEG) and/or by further blood tests (e.g., looking at other markers such as troponin).

Thus, for example, described herein are methods of treating patients for acute myocardial infarction (AMI) by taking a sample (e.g., of blood), and using a solid-phase assay that binds Lp-PLA2, and detecting the amount of an HDL marker, such as in particular ApoA1, from the material bound to the solid phase, and when the amount of HDL marker (e.g., ApoA1) is below a threshold, treating the patient for AMI.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the patient matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating, detecting or monitoring acute myocardial infarction (AMI) comprising:
exposing a blood sample from a patient to a solid phase support onto which an Lp-PLA2-binding molecule is coupled;
incubating the blood sample with an ApoA1-binding molecule; and
detecting the level of ApoA1 from the solid phase.

2. A method of treating, detecting or monitoring acute myocardial infarction (AMI) comprising:
exposing a blood sample from a patient to a solid phase support onto which an Lp-PLA2-binding molecule is coupled;
incubating the solid phase support with an ApoA1-binding molecule;
washing the solid phase support;
detecting the level of ApoA1 from the solid phase; and
treating the patient for AMI if the amount of ApoA1 detected is below a threshold.

3. The method of claim 1, further comprising treating the patient for AMI if the level of ApoA1 is below a threshold.

4. The method of claim 1, further comprising treating the patient for AMI if the level of ApoA1 detected is below 0.7 ng/ml.

5. The method of claim 1, further comprising washing the solid phase support.

6. The method of claim 1, wherein exposing the blood sample from the patient to the solid phase support comprising adding the sample to a solid phase support to which an antibody or an antibody fragment specific for Lp-PLA2 is coupled.

7. The method of claim 1, wherein incubating the blood sample with the ApoA1-binding molecule comprises incubating with an antibody or an antibody fragment that binds ApoA1.

8. The method of claim 1, wherein detecting the level of ApoA1 from the solid phase support comprises reacting a horseradish peroxidase (HRP) bound to an antibody against ApoA1 to detect a signal and quantifying the detected signal.

9. A system for detecting or monitoring acute myocardial infarction (AMI), the system comprising:
a first component comprising an Lp-PLA2 binding molecule coupled to a solid-phase support;
a reagent comprising an ApoA1 binding molecule to which a detection moiety is coupled.

10. The system of claim 9, wherein the first component comprises a solid-phase support to which an antibody or antibody fragment that binds to Lp-PLA2 has been coupled.

11. The system of claim 9, wherein the reagent comprises a solution including an ApoA1 antibody or antibody fragment coupled to a detection moiety.

12. The system of claim 9, further comprising a set of standards configured to calibrate the level of ApoA1 detected by the detection moiety.

13. A system for detecting or monitoring acute myocardial infarction (AMI), the system comprising:
a first component comprising an LpPLA2 binding molecule coupled to a solid-phase support;
a first wash buffer;
a reagent comprising an ApoA1 binding molecule to which a detection moiety is coupled; and
a detection buffer.

14. The system of claim 13, wherein the first component comprises a solid-phase support to which an antibody or antibody fragment that binds to Lp-PLA2 has been coupled.

15. The system of claim 13, wherein the reagent comprises a solution including an ApoA1 antibody or antibody fragment coupled to a detection moiety.

16. The system of claim 13, further comprising a set of standards configured to calibrate the level of ApoA1 detected by the detection moiety.

17. The method of claim 2, further comprising treating the patient for AMI if the level of ApoA1 detected is below 0.7 ng/ml.

18. The method of claim 2, wherein exposing the blood sample from the patient to the solid phase support comprising adding the sample to a solid phase support to which an antibody or an antibody fragment specific for Lp-PLA2 is coupled.

19. The method of claim 2, wherein incubating the blood sample with the ApoA1-binding molecule comprises incubating with an antibody or an antibody fragment that binds ApoA1.

20. The method of claim 2, wherein detecting the level of ApoA1 from the solid phase support comprises reacting a horseradish peroxidase (HRP) bound to an antibody against ApoA1 to detect a signal and quantifying the detected signal.
